(19) United States
(12) Patent Application Publication
(10) Pub. No.: US 2023/0072060 A1
(43) Pub. Date: Mar. 9, 2023

(54) COMPUTER SYSTEM AND METHOD FOR CALCULATING PAYMENT AMOUNT

(71) Applicant: Hitachi, Ltd., Tokyo (JP)

(72) Inventor: Kunihiko KIDO, Tokyo (JP)

(21) Appl. No.: 17/690,282

(22) Filed: Mar. 9, 2022

(30) Foreign Application Priority Data
Sep. 9, 2021 (JP) 2021-146637

(51) Int. Cl. G16H 50/20; G06Q 30/02

(52) U.S. Cl. CPC G16H 50/20; G06Q 30/0283

(57) ABSTRACT

A computer system calculates an execution probability of a cell therapy using therapy result data, generate a second model by incorporating an evaluation model for evaluating deterioration over time of a stored cells into a first model, the first model is a model for calculating a success probability of the cell therapy, executes a simulation of estimating a profit of a business operator due to success of the cell therapy by varying a storage period of the cells in a storage facility and a payment amount of the business operator to the storage facility using the execution probability of the cell therapy and the second model, and calculates an optimum storage period and an optimum payment amount that maximize the profit of the business operator based on the result of the simulation.

### CLAIM OF PRIORITY

The present application claims priority from Japanese patent application JP 2021-146637 filed on Sep. 9, 2021, the content of which is hereby incorporated by reference into this application.

### BACKGROUND OF THE INVENTION

The present invention relates to techniques for determining the cost and duration of storage of cells used for therapy.

In the medical fee claim and payment system, the payment from the payment fund after claiming medical fees may be delayed, and the management of medical institutions may deteriorate due to cash flow. In addition, medical office work is inefficient, and there are problems with data storage, security, and the like. On the other hand, the techniques described in JP 2002-230298 A and JP 2002-251458 A are known.

JP 2002-230298 A discloses a technique in which “a business operator intervenes in a financial contract between a medical institution and a financial institution for a medical fee credit claimed to a payment fund, and the business operator provides various services such as application software network distribution service for medical fee statements, for example, for a smooth operation thereof.”

JP 2002-251458 A discloses “a medical expense optimization system including a step of outputting a standard medical fee and a patient's copayment amount associated with a selected optimum treatment method, a step of outputting a standard upper limit of a drug expense portion associated with the medical fee, and a step of outputting a drug, especially a generic drug, that can be prescribed within a range without exceeding the standard upper limit of the drug expense portion and a patient's copayment amount associated with the drug.”

### SUMMARY OF THE INVENTION

In cell therapy using autologous cells such as CAR-T therapy, a therapy is performed in a procedure in which cells are collected from a patient at a medical institution, transported to a cell processing center in an ultra-low temperature container, and processed at the cell processing center, and the processed cells are returned to the medical institution and are transfused into the patient.

At this time, it takes 1 to 3 months for transportation, processing, and transfusion, and there are cases where the patient's condition deteriorates and he/she dies during this waiting time. In addition, it is common to perform strong anticancer drug treatment before cell therapy, and there are cases where cell processing fails or sufficient therapeutic effect cannot be obtained owing to cell deterioration due to anticancer drug treatment. In the case of failures in cell processing and therapy, the pharmaceutical company suffers a loss since there is no reimbursement related to the treatment or therapy.

Therefore, it is considered that when cells are collected and stored in a frozen state before the patient is treated with a strong anticancer drug treatment, the success rate of cell processing and the therapeutic effect can be improved, the loss of the pharmaceutical company can be suppressed, and the survival rate of patients can be improved. However, the storage cost of cells may not be reimbursed by medical fees, and there is a problem that patients and pharmaceutical companies must bear the storage cost.

A representative example of the present invention disclosed in this specification is as follows: a computer system comprises a computer having a processor, a storage device coupled to the processor, and a network interface coupled to the processor. The computer system is accessible to a storage facility for storing cells collected from a patient, a processing facility for processing the stored cells, and a medical facility for performing cell therapy using the processed cells. A storage fee, according to a storage period, paid to the storage facility being borne by the patient and a business operator who operates the processing facility. The business operator receives a success reward for the cell therapy from the patient. The processor is configured to: calculate an execution probability of the cell therapy using therapy result data managed by the medical facility, and store the execution probability of the cell therapy in the storage device; generate a second model by incorporating an evaluation model for evaluating deterioration over time of the stored cells into a first model, and store the second model in the storage device, the first model is a model for calculating a success probability of the cell therapy, generated using characteristics of the cells collected from the patient who has undergone the cell therapy and the therapy result data of the patient; execute a simulation of estimating a profit of the business operator due to success of the cell therapy by varying a storage period of the cells in the storage facility and a payment amount of the business operator to the storage facility using the execution probability of the cell therapy and the second model, and store a result of the simulation in the storage device; and calculate an optimum storage period and an optimum payment amount that maximize the profit of the business operator based on the result of the simulation.

According to the present invention, the collected cells can be stored based on a reasonable payment amount from the viewpoint of the profit of pharmaceutical companies. Other problems, configurations, and effects than those described above will become apparent in the descriptions of embodiments below.

### BRIEF DESCRIPTION OF THE DRAWINGS

The present invention can be appreciated by the description which follows in conjunction with the following figures, wherein:

FIG. 1 is an image diagram of the service of the present invention;

FIG. 2 is a diagram showing a configuration example of a system that realizes the present invention;

FIG. 3 is a diagram showing an example of a state transition model of a patient who uses a medical institution of a first embodiment;

FIG. 4 is a flowchart illustrating an example of a discrimination model generation process executed by a cell storage management system of the first embodiment;

FIG. 5 is a flowchart illustrating an example of cost calculation process executed by the cell storage management system of the first embodiment;

FIG. 6 is a diagram showing an example of a copayment amount acceptance curve held by the cell storage management system of the first embodiment;

FIG. 7 is a diagram showing an example of a history distribution calculated by the cell storage management system of the first embodiment; and

FIG. 8 is a diagram showing the breakdown of a storage fee paid to a frozen storage facility of the first embodiment.

### DETAILED DESCRIPTION OF INVENTION

Now, a description is given of an embodiment of this invention referring to the drawings. It should be noted that this invention is not to be construed by limiting the invention to the content described in the following embodiment. A person skilled in the art would easily recognize that a specific configuration described in the following embodiment may be changed within the scope of the concept and the gist of this invention.

In a configuration of this invention described below, the same or similar components or functions are assigned with the same reference numerals, and a redundant description thereof is omitted here.

Notations of, for example, “first”, “second”, and “third” herein are assigned to distinguish between components, and do not necessarily limit the number or order of those components.

The position, size, shape, range, and others of each component illustrated in, for example, the drawings may not represent the actual position, size, shape, range, and other metrics in order to facilitate understanding of this invention. Thus, this invention is not limited to the position, size, shape, range, and others described in, for example, the drawings.

### First Embodiment

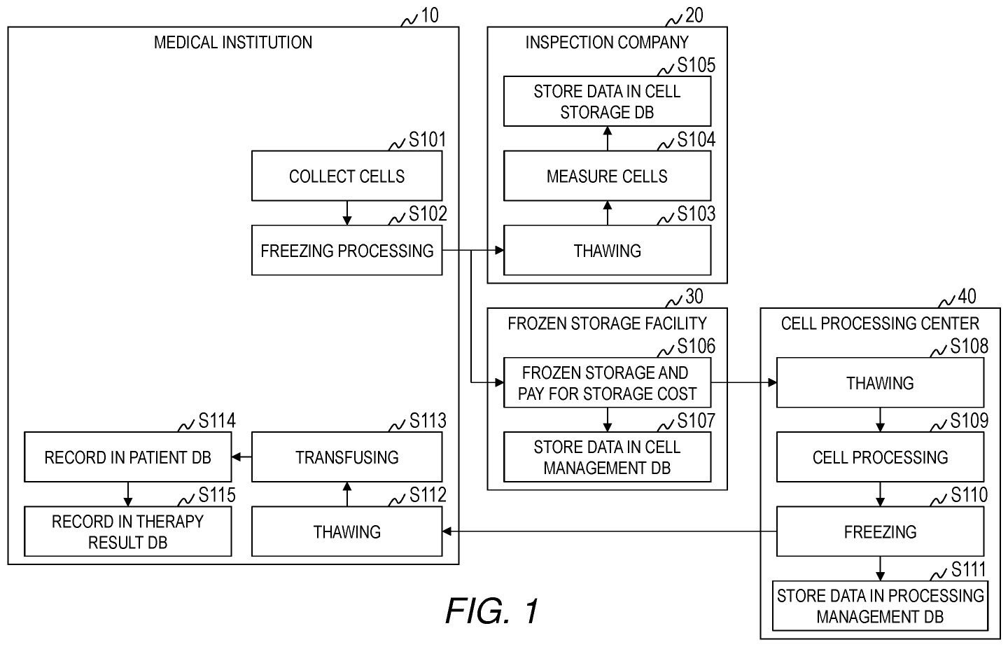

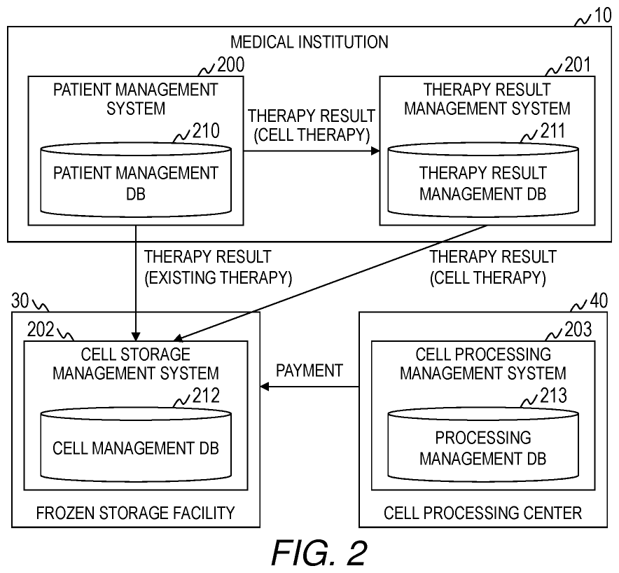

FIG. 1 is an image diagram of the service of the present invention. FIG. 2 is a diagram showing a configuration example of a system that realizes the present invention.

As stakeholders of a service, there are a medical institution 10, an inspection company 20, a frozen storage facility 30, and a cell processing center 40.

The medical institution 10 is a facility that treats a patient, such as a hospital. As shown in FIG. 2, the medical institution 10 operates a patient management system 200 and a therapy result management system 201.

The patient management system 200 is a system for managing patients who use the medical institution 10, and holds a patient management DB 210. The patient management DB 210 stores patient data for managing patients. The patient data includes patient's personal information (name, age, gender, and the like), a patient ID, a medical condition, therapy performed, therapy result, vital signs, and the like. The therapy result management system 201 is a system for managing the results of the therapy performed on the patient, and holds a therapy result management DB 211. The therapy result management DB 211 stores the therapy result data for managing the result of the therapy performed on the patient. The therapy result data includes a patient ID, the content of the therapy, the result of the therapy, and the like. In a first embodiment, the therapy result data related to the cell therapy is accumulated in the therapy result management DB 211.

The inspection company 20 is a company that inspects the cells collected from patients.

The frozen storage facility 30 is a facility for freezing and storing cells. The frozen storage facility 30 operates the cell storage management system 202.

The cell storage management system 202 is a system for managing frozen cells and holds a cell management DB 212. The cell management DB 212 stores cell data for managing frozen cells. The cell data includes a patient ID, the date and time when the frozen storage was started, the cell inspection result, and the like.

The cell processing center 40 is a facility for processing the cells collected from patients. In the present specification, it is assumed that the cell processing center 40 is operated by a pharmaceutical company. However, the cell processing center 40 may be operated by companies such as Contract Development and Manufacturing Organization (CDMO). The cell processing center 40 manages a cell processing management system 203. The cell processing management system 203 is a system for managing processed cells, and holds a processing management DB 213. The processing management DB 213 stores processing data for managing the processed cells. The processing data includes a patient ID, processing date, processing content, and the like.

The patient management system 200, the therapy result management system 201, the cell storage management system 202, and the cell processing management system 203 are configured of at least one computer. The computer has a processor, a main storage device, an auxiliary storage device, and a network interface. In addition, each system may include a storage device and a network switch.

Here, the service flow will be described.

The medical institution 10 collects cells from a patient (step S101) and freezes the cells at about −80 degrees (step S102). The records of cell collection are registered in patient management DB 210. The cells are managed in association with the patient ID. The method for collecting cells is, for example, collection of white blood cells by apheresis. However, the present invention is not limited to the method for collecting cells.

The frozen cells are stored in a container such as a dry shipper and transported. Specifically, the medical institution 10 sends a portion of the frozen cells to the inspection company 20, and sends the remaining cells to the frozen storage facility 30.

The inspection company 20 thaws a portion of the cells taken out from the container (step S103) and measures the cells (step S104). In cell measurement, the characteristics of cells are measured. For example, measurement of cell surface protein by flow cytometry, or measurement of gene mutation and gene expression by Next Generation Sequencer (NGS) is performed. The present invention is not limited to the measuring method and the measurement items.

The inspection company 20 registers the inspection result including the patient ID and the measurement data in the cell management DB 212 by transmitting the inspection result to the frozen storage facility 30 (step S105).

The frozen storage facility 30 stores the cells sent from the medical institution 10 (step S106). The frozen storage facility 30 registers the data regarding the cells to be stored in the cell management DB 212 (step S107). The pharmaceutical company bears a portion of the frozen storage fee (storage fee), and the patient bears a portion of the storage fee. The amount borne by the pharmaceutical company is described as a payment amount, and the amount borne by the patient is described as a copayment amount.

In a case where the frozen storage facility 30 receives a request for cell processing from the medical institution 10, the cells are stored in a container such as a dry shipper and sent to the cell processing center 40.

The cell processing center 40 thaws the cells (step S108) and performs cell processing (step S109). Further, the cell processing center 40 freezes the processed cells (step S110), and registers the data related to the processed cells in the processing management DB 213 (step S111). The cell processing center 40 stores the processed cells in a container such as a dry shipper and sends them to the medical institution 10.

The medical institution 10 thaws the processed cells (step S112) and transfuses the processed cells into the patient corresponding to the patient ID associated with the cells (step S113). The medical institution 10 registers the data regarding the response/non-response of the therapy using the processed cells in the patient management DB 210 (step S114). The patient management system 200 of the medical institution 10 collects the therapy result data related to the cell therapy together with the patient ID and transmits it to the therapy result management system 201. The therapy result management system 201 registers the received therapy result data together with the patient ID in the therapy result management DB 211 (step S115).

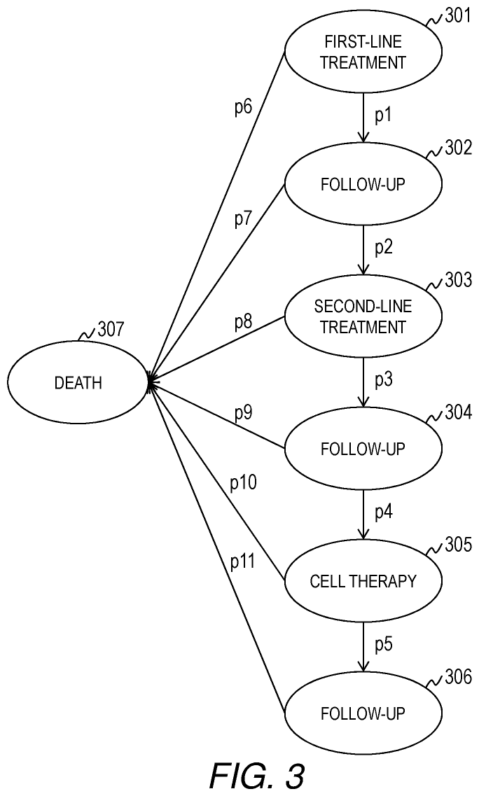

FIG. 3 is a diagram showing an example of a state transition model of a patient who uses the medical institution 10 of the first embodiment.

In the first embodiment, the state includes first-line treatment, second-line treatment, cell therapy, follow-up, and death. The first-line treatment and the second-line treatment represent treatments other than cell therapy using processed cells, such as anticancer drug treatment. Follow-up represents observation of progress after treatment.

As shown in FIG. 3, the patient's state transition model is represented as a graph with first-line treatment 301, follow-up 302, second-line treatment 303, follow-up 304, cell therapy 305, follow-up 306, and death 307 as nodes. The follow-up 302 represents the follow-up when the first-line treatment was performed, the follow-up 304 represents the follow-up when the second-line treatment was performed, and the follow-up 306 represents the follow-up when the cell therapy was performed. The directed edge connecting the states (nodes) represents the state transition, and a state transition probability pi is set. The index i is a natural number from 1 to 10.

The cell storage management system 202 of the first embodiment obtains the therapy result data from the therapy result management system 201. Moreover, the cell storage management system 202 obtains cell data from the cell management DB 212. In addition, the cell storage management system 202 calculates the payment amount that maximizes the profit based on the obtained therapy result data and cell data.

Specifically, the cell storage management system 202 generates a discrimination model for discriminating the response/non-response of the cell therapy using the therapy result data of the cell therapy and the cell data. The cell storage management system 202 calculates each state transition probability of the state transition model shown in FIG. 3 using the patient data and the discrimination model. The cell storage management system 202 calculates the profit distribution by performing a simulation using the state transition probability, and calculates the expected distribution of the patient's copayment amount. The cell storage management system 202 calculates the payment amount based on the profit distribution and the expected distribution of the copayment amount.

Next, the details of a method for calculating the payment amount will be described.

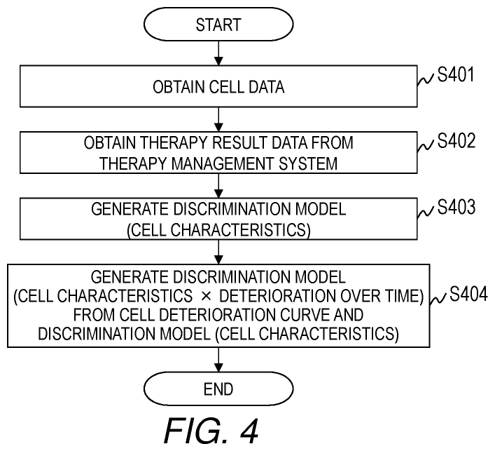

FIG. 4 is a flowchart illustrating an example of a discrimination model generation process executed by the cell storage management system 202 of the first embodiment.

In the first embodiment, it is assumed that a discrimination model is generated for each medical institution 10. However, a discrimination model common to all medical institutions 10 may be generated, or a discrimination model may be generated for each region. In this case, the range of data to be obtained may be changed.

The cell storage management system 202 obtains the cell data from the cell management DB 212 (step S401).

Next, the cell storage management system 202 obtains the therapy result data corresponding to the patient ID included in the cell data from the therapy result management system 201 (step S402).

Next, the cell storage management system 202 generates a discrimination model for discriminating the response/non-response of the cell therapy based on the cell data, by using the therapy result data and the characteristics of the cells (step S403).

For example, the cell storage management system 202 refers to therapy result data and labels the cell data as either response or non-response. The cell storage management system 202 generates a discrimination model by logistic regression analysis in which the label is the objective variable Y(i) and the characteristics of the cells included in the cell data are the explanatory variables X(j).

Here, the variable i is an integer from 1 to n, and the variable j is an integer from 1 to m. The integer n represents the number of patients and the Integer m represents the number of attributes included in the characteristics of the cell. In a case where the cell data contains gene expression, the number of attributes corresponds to the number of genes. In addition, by including the gene set involved in cell growth and cell death in the explanatory variable X(j), it becomes possible to better capture the characteristics of the cell.

For example, in logistic regression analysis, the coefficient b(j) and the constant b(0) of Formula (1) are calculated.

$$\begin{matrix}Formula ( 1 ) & \\ Y ( i ) ∼ \frac{1}{( 1 + exp ( - ( b ( 1 ) X ( 1 ) + … + b ( m ) X ( m ) + b ( 0 ) ) ) )} & ( 1 )\end{matrix}$$

The discrimination model is given by Formula (2). Z is the probability that the therapeutic effect is expected (response probability).

$$\begin{matrix}Formula ( 2 ) & \\ Z = \frac{1}{( 1 + exp ( - ( b ( 1 ) X ( 1 ) + … + b ( m ) X ( m ) + b ( 0 ) ) ) )} & ( 2 )\end{matrix}$$

The discrimination model may be generated using a method other than logistic regression analysis. For example, support vector machines, random forests, deep learning, and the like may be used.

Next, the cell storage management system 202 generates a discrimination model for discriminating the response/non-response of cell therapy based on cell characteristics, a freezing temperature, and a storage period by incorporating a model for evaluating cell deterioration over time of frozen cells into the discrimination model generated in step S403 (step S404). After that, the cell storage management system 202 saves the generated discrimination model and ends the discrimination model generation process.

Specifically, the cell storage management system 202 generates a new discrimination model by multiplying the discrimination model generated in step S403 by a cell deterioration curve. The cell deterioration curve is a curve representing the relationship between the freezing temperature and storage period of cells and the deterioration of cells, and is given by, for example, Formula (3). Here, the coefficients A and B are deterioration coefficients determined from the freezing temperature, and t is a variable representing the storage period.

$$\begin{matrix}Formula ( 3 ) & \\ B \times exp ( - \frac{t}{A} ) & ( 3 )\end{matrix}$$

Therefore, the discrimination model is given by Formula (4). W is the response probability.

$$\begin{matrix}Formula ( 4 ) & \\ W = Z \times B \times exp ( - \frac{t}{A} ) & ( 4 )\end{matrix}$$

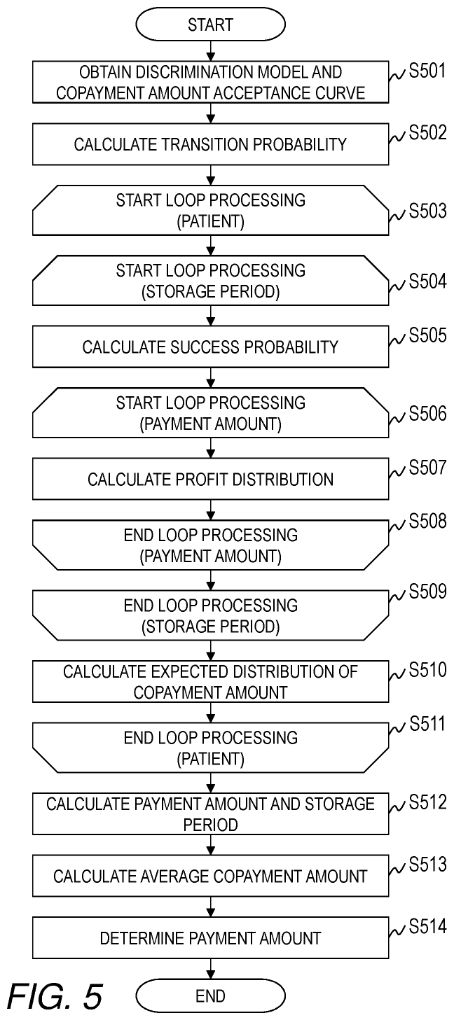

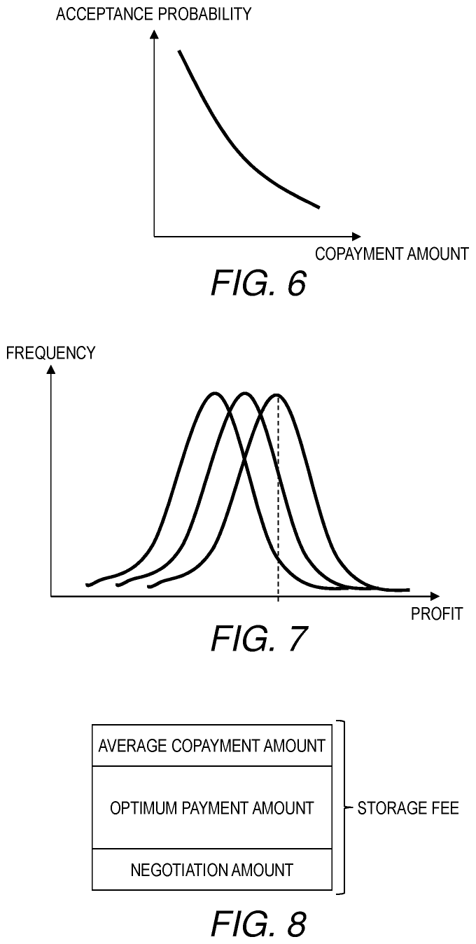

FIG. 5 is a flowchart illustrating an example of the cost calculation process executed by the cell storage management system 202 of the first embodiment. FIG. 6 is a diagram showing an example of a copayment amount acceptance curve held by the cell storage management system 202 of the first embodiment. FIG. 7 is a diagram showing an example of the history distribution calculated by the cell storage management system 202 of the first embodiment. FIG. 8 is a diagram showing the breakdown of the storage fee paid to the frozen storage facility 30 of the first embodiment.

In the first embodiment, it is assumed that the cost calculation process is executed for each medical institution 10. However, the cost calculation process may be executed for all medical institutions 10, or the cost calculation process may be executed for each region. In this case, the range of data to be obtained may be changed.

The cell storage management system 202 obtains the copayment amount acceptance curve and the discrimination model generated in the discrimination model generation process (step S501).

The copayment amount acceptance curve is a curve for calculating the probability that the patient will accept the copayment amount, and is given as, for example, a curve as shown in FIG. 6. The horizontal axis represents the copayment amount, and the vertical axis represents the acceptance probability. It is assumed that the copayment amount acceptance curve is set in advance.

Next, the cell storage management system 202 obtains the patient data from the patient management system 200, and uses the patient data to calculate transition probabilities p1, p2, p3, p4, p6, p7, p8, p9, and p11 (step S502). That is, the probability that cell therapy will be performed is calculated. The transition probability may be calculated by the patient management system 200.

Next, the cell storage management system 202 starts the loop processing for the patient (step S503). The cell storage management system 202 selects a target patient from the patient group managed by the cell management DB 212, and obtains the cell data of the target patient.

Next, the cell storage management system 202 starts the loop processing for the storage period (step S504). The cell storage management system 202 randomly selects a storage period. In the first embodiment, the storage period is selected in the range of 1 to 5 years. The change width is, for example, one month. In data processing, the storage period is treated as a number.

Next, the cell storage management system 202 calculates the response probability (step S505).

Specifically, the cell storage management system 202 calculates the response probability by setting the storage period selected in step S504 in the variable t of the discrimination model shown in Formula (4), and inputting the cell data. The response probability corresponds to the transition probability p5. Further, the cell storage management system 202 calculates the transition probability p10 by Formula (5).

Formula (5)

p10=1−p5 (5)

Through the above-described processing, the patient's state transition model is determined. In the first embodiment, the payment amount and the storage period that maximize the profit of the pharmaceutical company are estimated by the Monte Carlo simulation using the state transition model.

Next, the cell storage management system 202 starts the loop processing for the payment amount (step S506). The cell storage management system 202 randomly selects the payment amount within the range of 0 yen to the entire storage cost. The change width is, for example, 100,000 yen.

Next, the cell storage management system 202 calculates the profit distribution by performing a simulation based on the storage period, the transition probability, and the payment amount (step S507). The profit distribution is stored in association with the storage period and payment amount.

In the first embodiment, the profit distribution is calculated using the Monte Carlo simulation. For example, the cell storage management system 202 calculates the profit by integrating the cost and sales in a case where the state of 10,000 patients virtually transitions according to the state transition model, and calculates the profit distribution as shown in FIG. 7 by aggregating the profits of 10,000 patients.

In the state of any of the first-line treatment 301, follow-up 302, second-line treatment 303, and follow-up 304, if it is less than 5 years from the date of cell collection, the payment from a pharmaceutical company to the frozen storage facility 30 occurs but no sales are generated. In the state of any of the first therapy 301, follow-up 302, second-line treatment 303, and follow-up 304, if 5 years have passed from the date of cell collection, the cells are discarded due to deterioration, and the payment amount from the pharmaceutical company to the frozen storage facility 30 will be 0 yen. When the state of the first therapy 301, the follow-up therapy 302, and the follow-up therapy 303 transitions to the death 307, the payment amount becomes 0 yen, and the simulation ends at this point. In the state of the cell therapy 305, the cost of cell processing and the cost of cell transport between facilities are incurred. Further, if 5 years have passed from the date of cell collection until the state transitions to the first therapy 301, the follow-up 302, the second therapy 303, and the follow-up 304, the cells are discarded due to deterioration, so that it is necessary to collect cells again, which incurs costs. The state of the follow-up 306 means that the cell therapy did not fail, that is, the therapy was successful. Therefore, when the state transitions from the cell therapy 305 to the follow-up 306, the pharmaceutical company can be reimbursed for the treatment cost from the patient for the first time, which can be recorded as sales.

By the above-described Monte Carlo simulation, the profit distribution is calculated for each combination of the payment amount and the storage period.

Next, the cell storage management system 202 determines whether or not to end the loop processing for the payment amount (step S508).

In a case where it is determined that the loop processing for the payment amount is not to be ended, the cell storage management system 202 returns to step S506 and executes the same processing.

In a case where it is determined that the loop processing for the payment amount is to be ended, the cell storage management system 202 determines whether or not to end the loop processing for the storage period (step S509).

In a case where it is determined that the loop processing for the storage period is not to be ended, the cell storage management system 202 returns to step S504 and executes the same processing.

In a case where it is determined that the loop processing for the storage period is to be ended, the cell storage management system 202 calculates the expected distribution of the copayment amount using the copayment amount acceptance curve (step S510).

Specifically, the cell storage management system 202 calculates the copayment amount from the payment amount selected in the simulation, and calculates the acceptance probability using the copayment amount and the copayment amount acceptance curve. Further, the cell storage management system 202 calculates the product of the acceptance probability and the copayment amount. By executing the same processing for each payment amount, the expected distribution of the copayment amount is calculated.

Next, the cell storage management system 202 determines whether or not to end the loop processing for the patient (step S511).

In a case where it is determined that the loop processing for the patient is not to be ended, the cell storage management system 202 returns to step S503 and performs the same process.

In a case where it is determined that the loop processing for the patient is to be ended, the cell storage management system 202 calculates the optimum payment amount and optimum storage period based on the profit distribution (step S512).

Specifically, the cell storage management system 202 compares the average values of the respective profit distributions and selects the profit distribution that maximizes the average value. The cell storage management system 202 calculates the payment amount and storage period associated with the selected profit distribution as the optimum payment amount and optimum storage period. Thus, the optimum payment amount and storage period are the reasonable payment amount and storage period that maximize the profit of the pharmaceutical company.

Next, the cell storage management system 202 calculates the average value of the expected distribution of the copayment amount as an average copayment amount (step S513).

Next, the cell storage management system 202 determines the payment amount based on the storage fee, the optimum payment amount, the optimum storage period, and the average copayment amount (step S514). After that, the cell storage management system 202 ends the cost calculation process.

Specifically, the cell storage management system 202 calculates a negotiation amount using Formula (6). The breakdown of the storage fee is shown in FIG. 8.

Formula (6)

Negotiation amount=Storage fee−Optimum payment amount−Average copayment amount (6)

The pharmaceutical company determines the negotiation amount by comprehensively judging the business condition and the medical condition of the patient, and the like, and determines the payment amount that maximizes the total of the negotiation amount and the profit.

According to the present invention, pharmaceutical companies can store collected cells at a cost that maximizes profits. Further, since the therapy result is improved, the patient's Quality of Life (QoL) is improved.

Although the cell storage management system 202 executes various processes, other systems may execute the processes. Further, a system which is not shown may execute the processes.

The present invention can also be applied to a service for storing cells by a method other than freezing.

The present invention is not limited to the above embodiment and includes various modification examples. In addition, for example, the configurations of the above embodiment are described in detail so as to describe the present invention comprehensibly. The present invention is not necessarily limited to the embodiment that is provided with all of the configurations described. In addition, a part of each configuration of the embodiment may be removed, substituted, or added to other configurations.

A part or the entirety of each of the above configurations, functions, processing units, processing means, and the like may be realized by hardware, such as by designing integrated circuits therefor. In addition, the present invention can be realized by program codes of software that realizes the functions of the embodiment. In this case, a storage medium on which the program codes are recorded is provided to a computer, and a CPU that the computer is provided with reads the program codes stored on the storage medium. In this case, the program codes read from the storage medium realize the functions of the above embodiment, and the program codes and the storage medium storing the program codes constitute the present invention. Examples of such a storage medium used for supplying program codes include a flexible disk, a CD-ROM, a DVD-ROM, a hard disk, a solid state drive (SSD), an optical disc, a magneto-optical disc, a CD-R, a magnetic tape, a non-volatile memory card, and a ROM.

The program codes that realize the functions written in the present embodiment can be implemented by a wide range of programming and scripting languages such as assembler, C/C++, Perl, shell scripts, PHP, Python and Java.

It may also be possible that the program codes of the software that realizes the functions of the embodiment are stored on storing means such as a hard disk or a memory of the computer or on a storage medium such as a CD-RW or a CD-R by distributing the program codes through a network and that the CPU that the computer is provided with reads and executes the program codes stored on the storing means or on the storage medium.

In the above embodiment, only control lines and information lines that are considered as necessary for description are illustrated, and all the control lines and information lines of a product are not necessarily illustrated. All of the configurations of the embodiment may be connected to each other.

What is claimed is:

1. A computer system comprising:
a computer having a processor, a storage device coupled to the processor, and a network interface coupled to the processor,
the computer system being accessible to a storage facility for storing cells collected from a patient, a processing facility for processing the stored cells, and a medical facility for performing cell therapy using the processed cells,
a storage fee, according to a storage period, paid to the storage facility being borne by the patient and a business operator who operates the processing facility,
the business operator receiving a success reward for the cell therapy from the patient,
the processor being configured to:
calculate an execution probability of the cell therapy using therapy result data managed by the medical facility, and store the execution probability of the cell therapy in the storage device;
generate a second model by incorporating an evaluation model for evaluating deterioration over time of the stored cells into a first model, and store the second model in the storage device, the first model is a model for calculating a success probability of the cell therapy, generated using characteristics of the cells collected from the patient who has undergone the cell therapy and the therapy result data of the patient;
execute a simulation of estimating a profit of the business operator due to success of the cell therapy by varying a storage period of the cells in the storage facility and a payment amount of the business operator to the storage facility using the execution probability of the cell therapy and the second model, and store a result of the simulation in the storage device; and
calculate an optimum storage period and an optimum payment amount that maximize the profit of the business operator based on the result of the simulation.

2. The computer system according to claim 1, wherein the processor is configured to calculate a profit distribution of the business operator for each combination of the storage period and the payment amount in the simulation, and
the processor is configured to calculate the optimum storage period and the optimum payment amount based on a plurality of profit distributions.

3. The computer system according to claim 2, wherein
the storage device stores, as the evaluation model, a cell deterioration evaluation function in which the storage period is used as a variable,
the first model is a function in which the characteristics of the cells are variables, and
the processor is configured to generate the second model by multiplying the first model by the evaluation model.

4. The computer system according to claim 3, wherein
the storage device stores a probability model for calculating a probability that the patient will accept a copayment amount based on the copayment amount of the patient for the storage facility,
the processor is configured to:
calculate, by using the probability model, an expected distribution of the copayment amount in a case where the copayment amount is varied, and store the expected distribution of the copayment amount in the storage device;
calculate an average copayment amount of the patient using the expected distribution of the copayment amount, and store the average copayment amount of the patient in the storage device; and
calculate the payment amount of the business operator based on the storage fee, the optimum payment amount, and the average copayment amount.

5. The computer system according to claim 2, wherein
the characteristic of the cell is at least one of a gene mutation, gene expression, and a gene set involved in cell growth and cell death.

6. A method for calculating a payment amount related to cell therapy using cells collected from a patient to be executed by a computer system,
the computer system including: a computer having a processor, a storage device coupled to the processor, and a network interface coupled to the processor,
the computer system being accessible to a storage facility for storing cells collected from a patient, a processing facility for processing the stored cells, and a medical facility for performing cell therapy using the processed cells,
a storage fee, according to a storage period, paid to the storage facility being borne by the patient and a business operator who operates the processing facility,
the business operator receiving a success reward for the cell therapy from the patient,
the method for calculating the payment amount including:
a first step of calculating, by the processor, an execution probability of the cell therapy using therapy result data managed by the medical facility, and storing the execution probability of the cell therapy in the storage device;
a second step of generating, by the processor, a second model by incorporating an evaluation model for evaluating deterioration over time of the stored cells into a first model, and storing the second model in the storage device, the first model is a model for calculating a success probability of the cell therapy, generated using characteristics of the cells collected from the patient who has undergone the cell therapy and the therapy result data of the patient;
a third step of executing, by the processor, a simulation of estimating a profit of the business operator due to success of the cell therapy by varying a storage period of the cells in the storage facility and a payment amount of the business operator to the storage facility using the execution probability of the cell therapy and the second model, and storing a result of the simulation in the storage device, and
a fourth step of calculating, by the processor, an optimum storage period and an optimum payment amount that maximize the profit of the business operator based on the result of the simulation.

7. The method for calculating the payment amount according to claim 6, wherein
the third step includes a step of calculating, by the processor, a profit distribution of the business operator for each combination of the storage period and the payment amount, and
the fourth step includes a step of calculating, by the processor, the optimum storage period and the optimum payment amount based on a plurality of profit distributions.

8. The method for calculating the payment amount according to claim 7, wherein
the storage device stores, as the evaluation model, a cell deterioration evaluation function in which the storage period is used as a variable,
the first model is a function in which the characteristics of the cells are variables, and
the second step includes a step of generating, by the processor, the second model by multiplying the first model by the evaluation model.

9. The method for calculating the payment amount according to claim 8, wherein
the storage device stores a probability model for calculating a probability that the patient will accept a copayment amount based on the copayment amount of the patient for the storage facility, and
the method of calculating the payment amount includes:
a step of calculating, by the processor, by using the probability model, an expected distribution of the copayment amount in a case where the copayment amount is varied, and storing the expected distribution of the copayment amount in the storage device;
a step of calculating, by the processor, an average copayment amount of the patient using the expected distribution of the copayment amount, and storing the average copayment amount of the patient in the storage device, and
a step of calculating, by the processor, the payment amount of the business operator based on the storage fee, the optimum payment amount, and the average copayment amount.

10. The method for calculating the payment amount according to claim 7, wherein
the characteristic of the cell is at least one of a gene mutation, gene expression, and a gene set involved in cell growth and cell death.
